(19) United States
(12) Patent Application Publication
(10) Pub. No.: US 2026/0074337 A1
(43) Pub. Date: Mar. 12, 2026

(54) BATTERY CLIP

(71) Applicant: Roche Diabetes Care, Inc., Indianapolis, IN (US)

(72) Inventor: Michel Alain Jean Cadio, Carmel, IN (US)

(73) Assignee: Roche Diabetes Care, Inc., Indianapolis, IN (US)

(21) Appl. No.: 19/392,916

(22) Filed: Nov. 18, 2025

Related U.S. Application Data
(63) Continuation of application No. PCT/US2023/068324, filed on Jun. 13, 2023.

(51) Int. Cl. H01M 50/202; A61B 5/00; A61B 5/155; H01M 50/224; H01M 50/244; H01M 50/247

(52) U.S. Cl. CPC H01M 50/202; A61B 5/155; A61B 5/4839; H01M 50/224; H01M 50/244; H01M 50/247; A61B 2560/0214

(57) ABSTRACT

A battery clip for retaining a battery to a circuit carrier is disclosed along with an assembly including a circuit carrier having a plurality of flexible tabs. The battery clip includes a plurality of slots configured to mate with and receive the flexible tabs of the circuit carrier such that the battery clip and the circuit carrier are secured together such that a first terminal of a retained battery physically contacts and is in electrical communication with the circuit carrier's first pad, the battery clip further comprising one or more flexible conductive fingers configured to contact a second terminal of a retained battery to establish an electrical communication between the fingers and the second terminal, and wherein the battery clip includes an electrically conductive material and establishes electrical communication between the fingers and the circuit carrier's second pad.

### CROSS-REFERENCE TO RELATED APPLICATIONS

This application claims priority to PCT application number PCT/US2023/068324 filed Jun. 13, 2023. The full disclosure of the PCT application number PCT/US2023/068324 is incorporated by reference herein.

### FIELD

The present disclosure relates generally to a battery clip, a circuit carrier assembly, a medical device for detecting at least one analyte in a body fluid and to a method for assembling a circuit carrier assembly. The medical device may be applied in the field of continuous monitoring of an analyte in a body fluid of a user, specifically in the field of home care and in the field of professional care, such as in hospitals. Other applications, however, are also feasible.

### BACKGROUND

Monitoring certain body functions, more particularly monitoring one or more concentrations of certain analytes such as blood glucose, plays an important role in the prevention and treatment of various diseases. Blood glucose monitoring, besides by using optical measurements, specifically may be performed by using electrochemical biosensors. In addition to so-called spot measurements, in which a sample of a body fluid is taken from a user in a targeted fashion and examined with respect to the analyte concentration PCT/US2023/06832 continuous measurements are increasingly being utilized. Thus, in the recent past, continuous measuring of glucose in the interstitial tissue (also referred to as continuous glucose monitoring or CGM) for example has been established as another important method for managing, monitoring and controlling diabetes.

In the process, an active sensor region is applied directly to a measurement site, which is generally arranged in an interstitial tissue, and, for example, converts glucose into electrical charge by using an enzyme (e.g. glucose oxidase, GOD), which charge is related to the glucose concentration and can be used as a measurement variable. Examples of such transcutaneous measurement systems are described in U.S. Pat. No. 6,360,888 B1 or in US 2008/0242962 A1.

Hence, current continuous glucose monitoring systems typically are transcutaneous systems or subcutaneous systems. This means that the actual sensor or at least a measuring portion of the sensor is arranged under the skin of the user. However, an evaluation and control part of the system (also referred to as a patch) is generally situated outside of the body of the user, outside of the human or animal body. In the process, the sensor is generally applied using an insertion instrument, which is likewise described in U.S. Pat. No. 6,360,888 B1 in an exemplary fashion. Other types of insertion instruments are also known.

The sensor typically comprises a substrate, such as a flat substrate, onto which an electrically conductive pattern of electrodes, conductive traces and contact pads may be applied. In use, the conductive traces typically are isolated by using one or more electrically insulating materials. The electrically insulating material typically further also acts as a protection against humidity and other detrimental substances and, as an example, may comprise one or more cover layers such as resists.

For the purpose of operating the patch, power may be provided by a coin battery such as a CR 1220. This coin battery may be electrically connected to a printed circuit board assembly (PCBA) of the patch and may be configured for supplying power to the patch during a 14-day wearing period of the continuous monitoring system. To ensure a signal integrity, an electromechanical interface is subject to special requirements. No contact interruptions should occur due to patient movements. A contact transition resistance is commonly influenced by various parameters. A major influencing factor is a contact normal force. Current solutions exemplarily provide for single-sided contacting such as with three contact points. However, such solutions commonly show the following disadvantages: one-sided contacting may lead to asymmetrical mechanical stresses. These asymmetrical mechanical stresses may be particularly critical for PCBA tracks and/or components. Further, a frictional connection may be made via a plastic housing of the patch and may create a long tolerance chain, which in turn may lead to higher contact normal forces to compensate for the tolerances. This may lead to a deformation of the plastic housing. Further, the contact normal forces may be different at each of the three contact points. There may be a direct dependency between different contact points. Thus, an independent adjustment of the contact normal force may not be possible. Further, plastic pins within the housing may be required. However, heat staking of the plastic pins is commonly a complex process that introduces thermal stresses.

U.S. Pat. No. 10,332,623B2 describes a medicament delivery device including a battery clip for a medical injector.

U.S. Pat. No. 10,765,369B2 describes a simple, disposable sensing device for sensing an analyte housed in a single case. The sensing device can transmit sensor data to monitoring device(s). The sensing device includes: a case having a lower major wall adapted to be mounted against a patient's skin, and an upper opposing major wall; a sensor extending from the case and having a distal end sensitive to the analyte to produce an electrical signal, and a proximal end within the case having electrical contacts; a printed circuit board assembly within the case supported by one of the major walls to receive the electrical signal via the electrical contacts; and an elastomeric pad disposed in the case and biased by the other major wall to urge the proximal end of the sensor into contact with the printed circuit board assembly and maintain an electrical connection between the electrical contacts and the printed circuit board assembly.

US20210067188A1 describes a wearable device having a horizontally polarized antenna and a vertically polarized antenna to gain the benefit of both types of polarization resulting in optimal signal transmission to and reception by a user's smartphone or mobile device. The wearable device includes a printed circuit board on a first plane along which plane the signal from the horizontally polarized signal will propagate. The printed circuit board includes a conductive ground plane and a trace antenna conductively coupled on one end of the trace to the conductive ground plane between which the horizontally polarized field is generated when the trace antenna is excited. A vertical field enhancer, parallel to the first plane and a distance from the trace antenna, is coupled to the ground plane, such that when the trace antenna is excited, a vertically polarized field is generated between the trace antenna and the vertical field enhancer.

US20190083715A1 describes an electronic assembly which includes a casing to conduct electricity flowing between at least some electronic components disposed within the casing. The electronic components include an ultrasonic transducer coupled to emit ultrasonic signals, and a battery to provide the electricity. A controller is coupled to the ultrasonic transducer and the battery, and the controller includes logic that when executed by the controller causes the electronic assembly to perform operations, including: instructing the first ultrasonic transducer to emit the ultrasonic signals.

Despite the advantages achieved by the above-mentioned devices, several technical challenges remain. For example, force may be provided to the battery from one direction only. Further, protrusions from housings are required to fixedly secure a battery clip.

It is therefore desirable to provide a battery clip, a circuit carrier assembly, a medical device for detecting at least one analyte in a body fluid and a method for assembling a circuit carrier assembly which at least partially address the above-mentioned technical challenges. Specifically, a battery clip, a circuit carrier assembly, a medical device for detecting at least one analyte in a body fluid and a method for assembling a circuit carrier assembly are desirable which allow a reliable contacting and holding of a battery on a circuit carrier.

### BRIEF SUMMARY

This section provides a general summary of the disclosure, and is not a comprehensive disclosure of its full scope or all its features. Further areas of applicability and aspects of the disclosure will become apparent from the claims, the figures and the description provided herein. The description in this summary is intended for purposes of illustration and is not intended to limit the scope of the present disclosure.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, notwithstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

At least one of the above-described issues/problems is solved by a battery clip according to independent claim 1 as well as by a method for retaining a battery in a medical device according to independent claim 17. Preferred embodiments of the disclosure which may be realized in an isolated way or in any arbitrary combination are disclosed in the dependent claims and throughout the specification and the figures.

The disclosed battery clip is less expensive to manufacture than other battery clips where a non-standard battery with battery tabs is soldered to a circuit carrier and also provides for a small increase in thickness for an assembly with a circuit carrier and overall device as compared to a circuit carrier with a soldered battery. Further, even though the disclosed battery clip is not typically used with a battery that will be replaced, the disclosed battery clip and assembly do allow for an easy replacement of a battery since the battery is not soldered to the circuit carrier which advantageously allows the user to use the device beyond the lifetime of an individual battery. Further, the disclosed assembly allows for more options in the procurement of the battery (more available suppliers) making it easier to secure the availability of the battery.

A first aspect of the present disclosure relates to a battery clip for retaining at least one battery to at least a first battery contact pad of a circuit carrier, wherein the battery clip comprises at least one housing comprising an electrically conductive material, wherein the housing comprises opposing side walls connected to a bottom wall and at least one flexible conductive finger forming a top wall, the housing comprising at least one receptacle for at least partially receiving the circuit carrier and the battery, wherein the receptacle comprises at least one opening through which the battery and the circuit carrier are at least partially insertable, the at least one flexible conductive finger and the bottom wall being configured for retaining the battery to the first battery contact pad of the circuit carrier, wherein the at least one flexible conductive finger is configured for contacting the battery being arranged on a first side of the circuit carrier being inserted into the receptacle, wherein the bottom wall is configured for contacting a second side of the circuit carrier being inserted into the receptacle, the second side opposing the first side, and wherein the battery clip is configured to press on the battery to cause a first terminal of the battery to physically contact and be in electrical communication with the first battery contact pad.

The term “circuit carrier” as used herein is a broad term and is to be given its ordinary and customary meaning to a person of ordinary skill in the art and is not to be limited to a special or customized meaning. The term specifically relates to an arbitrary body provided for carrying at least one electronic, electrical, and/or optical element, in particular a plurality of such elements, wherein the body is designed to mechanically support and electrically connect the electronic, electrical, and/or optical elements. Specifically, the circuit carrier may be a planar circuit carrier. The term “planar” refers to a property of a body which comprises extensions in two dimensions, typically denoted as “surface” of the planar body, which exceed the extension in a third dimension, usually denoted as “thickness” of the planar body, by a factor of 2, at least a factor of 5, at least a factor of 10, or even at least a factor of 20 or more.

Specifically, the circuit carrier may be or may comprise a printed circuit board, usually abbreviated as “PCB”, which refers to an electrically non-conductive, planar substrate, also denoted as “board”, on which at least one sheet of an electrically conductive material, in particular a copper layer, is applied, specifically laminated, to the substrate, and which, in addition, comprises one or more electronic, electrical, and/or optical elements. In the PCB, the electrically insulating substrate may comprise a glass epoxy, wherein a cotton paper impregnated with a phenolic resin, typically tan or brown, may also be used as a substrate material. Depending on a number of sheets, the printed circuit board may be a single-sided PCB, a two-layer or double-sided PCB, or a multi-layer PCB, wherein different sheets may be connected with each other by using so-called “vias”.

Electrically conductive patterns or structures, such as tracks, traces, pads, vias for generating connections between adjacent sheets, or features such as solid conductive areas, may be introduced into the one or more sheets, preferably by removing a partition of the sheet, in particular by etching, silk screen printing, photoengraving, PCB milling, or laser resist ablation, at selected regions in the sheet, whereby the desired structures can be created. The etching can, preferably, be performed by using a photoresist material being coated onto the PCB which is, subsequently, exposed to light, whereby the desired pattern may be generated. Herein, the photoresist material may be adapted to protect the metal from dissolution into an etching solution. After etching, the PCB may be cleaned. By using this process, a particular PCB pattern can be mass produced. However, other kinds of separation processes or connection processes may also be applicable. By way of example, a track introduced into the PCB may function as a wire being fixed at a selected position, wherein adjacent tracks can be insulated from each other, on one hand, by the substrate material and, on the other hand, by an electrically insulating fluid under conditions at which the PCB is used, specifically by air or a protective gas which may be present in a gap between the adjacent tracks. In addition, a surface of the PCB may have a coating, also denoted as a solder resist, which may be designed for protecting the metal, specifically the copper, within the at least one sheet from detrimental environmental effects, such as corrosion, thus, reducing a chance that undesired short circuits may be generated by a solder or by stray bare wires. In a multi-layer PCB, only outer metal layers may be coated in this manner since inner metal layers are protected by the adjacent substrate layers.

Further, the electronic, electrical, and/or optical elements or components may be placed onto the substrate, such as by soldering, welding, or depositing, or, additionally or as an alternative, be embedded into the circuit carrier, such as by placing them into seats designated in the substrate for this purpose and/or by deliberately removing a partition of the circuit carrier. Preferably, surface mount components, specifically transistors, diodes, IC chips, resistors and capacitors, may, thus, be attached to the PCB by using electrical conductive leads which adjoin the respective component to metal tracks, traces, or areas on the same side of the substrate. As an alternative, through-hole mounting may be used, in particular, for extended or voluminous components, such as electrolytic capacitors or connectors. As a further alternative, the components may be embedded within the substrate.

The circuit carrier may be a flexible circuit carrier, specifically a flexible printed circuit board. The term “flexible printed circuit board” as used herein is a broad term and is to be given its ordinary and customary meaning to a person of ordinary skill in the art and is not to be limited to a special or customized meaning. The term specifically relates to an arbitrary printed circuit board having a flexible electrically non-conductive, planar substrate. The flexible printed circuit board may be configured to be variously bent and folded according to manufacturing needs of a particular application. The substrate of the flexible printed circuit board may specifically be made of polyester resin or polyimide resin. However, also other materials may be feasible.

The circuit carrier may be a rigid circuit carrier, specifically a rigid printed circuit board. The term “rigid printed circuit board” as used herein is a broad term and is to be given its ordinary and customary meaning to a person of ordinary skill in the art and is not to be limited to a special or customized meaning. The term specifically relates to an arbitrary printed circuit board having a rigid electrically non-conductive, planar substrate. The rigid printed circuit board may be inflexible in its structure and cannot be bent. Rigid materials which may be used for the substrate are fiber-enforced plastic materials such as fiber-enforced epoxy materials like glass-fiber-enforced epoxy materials such as FR-4. Other materials may be used.

In the field of circuit carriers (e.g., printed circuit boards (PCBs)), flexible printed circuit boards (flexible PCBs) have numerous advantages as compared to rigid printed circuit boards (rigid PCBs), including a capability of conforming to a desired shape (e.g., curved). Many small battery-operated electronic devices may benefit from having a flexible PCB. However, the one or more batteries and battery holders of such devices are typically the thickest part of an electronic assembly on a PCB and also tend to add rigidity to the PCB, defeating the purpose of having a flexible PCB. Accordingly, a need exists to provide battery power to small battery-operated electronic devices having a flexible PCB without adversely affecting the flexibility of the flexible PCB while providing as small a device thickness and footprint as possible. Typical battery (e.g., coin cell battery) mounting parts are made of a plastic housing and metal contacts and possibly some leads to connect to the electrical circuit. The housing and the leads use space and negatively impact the size of the device (making the device assembly bigger). Some other battery mountings rely on having tabs soldered to the battery and the tabs then soldered to the PCB but this solution calls for the usage of a non-standard battery making the supply chain more vulnerable to supplier issues and adding a soldering operation, thereby increasing manufacturing cycle time and thus increasing cost.

The term “side” as used herein is a broad term and is to be given its ordinary and customary meaning to a person of ordinary skill in the art and is not to be limited to a special or customized meaning. The term specifically relates to a surface of a body. As outlined above, the second side opposes the first side. As generally used, the term “opposing sides” refers to two surfaces, specifically to two planar surfaces, of a body, typically of a flat body. Specifically, the first side and the second side are not located within a common plane. The sides may specifically extend essentially parallel to each other. One of the sides may be referred to as a front side and another one of the sides may be referred to as a rear side. The first side may also be referred to as a battery-facing side.

As outlined above, the battery clip is configured for retaining the battery to first and second battery contact pads of the circuit carrier. The term “battery contact pad” as used herein is a broad term and is to be given its ordinary and customary meaning to a person of ordinary skill in the art and is not to be limited to a special or customized meaning. The term specifically relates to an arbitrary element having at least one electrically conductive surface designed for establishing an electrical contact between the battery and the circuit carrier. Specifically, the battery contact pad may comprise at least one layer of an electrically conductive material.

The term “battery clip” as used herein is a broad term and is to be given its ordinary and customary meaning to a person of ordinary skill in the art and is not to be limited to a special or customized meaning. The term specifically relates to an arbitrary element which is configured for holding a battery in place on an object which may specifically be a circuit carrier and for placing the battery in electrical communication with an electronic circuit system. Specifically, the battery clip may be configured for being fixed itself on the object, specifically on the circuit carrier. Further details on the battery clip are given below in more detail.

As outlined above, the battery clip comprises the at least one housing. The term “housing” as used herein is a broad term and is to be given its ordinary and customary meaning to a person of ordinary skill in the art and is not to be limited to a special or customized meaning. The term specifically relates to an arbitrary element which is configured for fully or partially enclosing at least interior space and for providing protection to the interior space, such as mechanical protection. Also other kinds of protection may be feasible. The housing may be configured for fully or partially encasing or surrounding an object being located at least partially in the interior space of the housing. The housing may specifically comprise at least one wall for fully or partially surrounding the interior space. The housing may specifically be an open housing. The housing may specifically have several sidewalls. The sidewalls may have one or more openings and/or recesses. The size of the battery clip typically depends on the type of battery used. For an example, if a CR1025 battery is used, the battery clip can be in one embodiment 10.5 mm wide by 8 mm long by 3.1 mm thick. As another example, if a SR920 battery is used, the battery clip can be in one embodiment 10.0 mm wide by 8 mm long by 2.7 mm thick. Thus, in typical embodiments, the battery clip can have a size only slightly bigger than the battery it is configured to hold, e.g., only 0.5 mm wider and 0.6 mm thicker than the battery.

The housing comprises at least one receptacle. The term “receptacle” as used herein is a broad term and is to be given its ordinary and customary meaning to a person of ordinary skill in the art and is not to be limited to a special or customized meaning. The term specifically relates to an arbitrary partially or fully enclosed space that may be usable to contain and/or store one or more objects. Specifically, the one or more objects may be at least partially receivable within the receptacle. Thus, the one or more objects may be at least partially encased or covered by walls of an element forming the receptacle. Optionally, the housing may comprise one or even more than one receptacle such as at least two receptacles. As outlined herein, the receptacle is configured for at least partially receiving the circuit carrier and the battery. Thus, the circuit carrier and the battery may respectively at least partially be encased by sidewalls of the housing forming the receptacle. Specifically one portion of the circuit carrier and/or of the battery may be located outside the receptacle and another portion of the circuit carrier and/or of the battery may be located inside the receptacle. The receptacle itself may be an open receptacle.

As outlined above, the receptacle comprises the at least one opening through which the battery and the circuit carrier are at least partially insertable. The term “opening” as used herein is a broad term and is to be given its ordinary and customary meaning to a person of ordinary skill in the art and is not to be limited to a special or customized meaning. The term specifically relates to an arbitrary hole or space such that one or more objects can pass through. The opening may be configured for providing access to the receptacle. The opening may be formed by or may be a recess of the sidewalls of the housing. The opening may have a shape such that the circuit carrier and the battery may respectively at least partially be insertable into the receptacle via the opening.

The battery clip, including its housing, may be made of at least one electrically conductive material. As generally used, the term “electrically conductive material” refers to a substance which is designed for conducting an electrical current through the substance. The electrically conductive material may, preferably, be stainless steel plated with nickel or be selected from a noble metal, especially gold; or from an electrically conductive carbon material. However, other kinds of electrically conductive materials may also be feasible. Preferably, at least one flexible conductive finger may comprise at least one layer of an electrically conductive material. Specifically, the contact surface of the at least one flexible conductive finger may be a coated surface. More specifically, a contact surface of the at least one flexible conductive finger may be at least partially coated with a noble metal such as with gold.

The term “flexible conductive finger” as used herein is a broad term and is to be given its ordinary and customary meaning to a person of ordinary skill in the art and is not to be limited to a special or customized meaning. The term specifically relates to an extending member which is electrically conductive and which may or may not be resilient or elastic but which can be bent or folded without breaking.

In another aspect of the disclosure, a method is provided for retaining a battery in a medical device, including providing a circuit carrier comprising a first battery contact pad, a second battery contact pad and a plurality of flexible tabs; and providing a battery clip configured to be secured to the circuit carrier, wherein the battery clip is configured to retain the battery for providing power to the circuit carrier such that the battery and the circuit carrier are pressed together such that a first terminal of the battery physically contacts and is in electrical communication with the first battery contact pad, the battery clip further comprising one or more flexible conductive fingers configured to contact a second terminal of the battery to establish an electrical communication between the one or more fingers and the second terminal of the battery, and wherein the battery clip comprises an electrically conductive material and establishes electrical communication between the one or more fingers and the second battery contact pad.

The term “flexible tab” as used herein is a broad term and is to be given its ordinary and customary meaning to a person of ordinary skill in the art and is not to be limited to a special or customized meaning. The term specifically relates to an extending member which may or may not be resilient or elastic but which can be bent or folded without breaking. Another aspect of the present disclosure relates to a circuit carrier assembly that includes the battery clip as described above and a circuit carrier comprising a first battery contact pad, a second battery contact pad and a plurality of flexible tabs wherein the battery clip is configured to clamp the battery and the circuit carrier together in a sandwich structure to securely hold the battery in the assembly and provide electrical communication between the battery and the circuit carrier.

The term “assembly” as used herein is a broad term and is to be given its ordinary and customary meaning to a person of ordinary skill in the art and is not to be limited to a special or customized meaning. The term specifically relates to a group of at least two elements which may interact with each other in order to fulfill at least one common function. The at least two components may be handled independently or may be coupled, connectable or integral in order to form a common device. The term “circuit carrier assembly” as used herein is a broad term and is to be given its ordinary and customary meaning to a person of ordinary skill in the art and is not to be limited to a special or customized meaning. The term specifically may refer, without limitation, to a group of a circuit carrier and of at least one another element which may interact with each other in order to fulfill at least one common function.

The circuit carrier assembly comprises at least one battery clip as described above or as will further be described below in more detail. Thus, for possible definitions and options of the battery clip, reference may be made to the disclosure of the battery clip according to the present invention.

Further, the circuit carrier assembly comprises at least one circuit carrier having at least a first battery contact pad and a second battery contact pad. The circuit carrier is at least partially insertable into the receptacle of the housing of the battery clip. For possible definitions and options of the circuit carrier, reference may be made to the disclosure of the circuit carrier according to the present invention.

The term “battery” as used herein is a broad term and is to be given its ordinary and customary meaning to a person of ordinary skill in the art and is not to be limited to a special or customized meaning. The term specifically relates to an arbitrary source of electric power comprising one or more electrochemical cells with external connections for powering an electrical device. When a battery supplies power, its positive terminal may be referred to as cathode and its negative terminal may be referred to as anode. The battery may specifically be a primary battery. The primary battery may be configured for being used once. The primary battery may also be referred to as a single-use or disposable battery. However, alternatively, the battery may be a secondary battery. The secondary battery may be configured for being discharged and recharged multiple times using an applied electric current. The secondary battery may also be referred to as a rechargeable battery.

The battery may specifically be a coin battery (i.e., a coin cell battery). The coin battery may have a planar shape. The term “planar” may refer to a property of a body which comprises extensions in two dimensions, typically denoted as “surface” of the planar body, which exceed the extension in a third dimension, usually denoted as “thickness” of the planar body, by a factor of 2, at least a factor of 5, at least a factor of 10, or even at least a factor of 20 or more. Specifically, the coin battery may be shaped as a cylinder typically 5 mm to 25 mm in diameter and 1 mm to 6 mm in high. The coin battery may resemble a button. Thus, the coin battery may also be referred to as button cell. A bottom body of the coin battery may form a positive terminal of the coin battery and a top cap may form a negative terminal of the coin battery. As an example, the battery may comprise a lithium 1025 coin cell battery.

As will be outlined below in more detail, the battery clip may exemplarily be applied in a medical device for detecting at least one analyte in a body fluid. The coin battery may be electrically connected to a printed circuit board of the medical device and may be configured for supplying power during a 14-day wearing period of the medical device.

As will be described in further detail below, the circuit carrier assembly may exemplarily be applied in a medical device for detecting at least one analyte in a body fluid. The medical device may specifically be configured for continuous monitoring of the analyte in the body fluid. The medical device may specifically be a single-use or disposable medical device. Thereby, an exchange of the battery may not necessarily be performed. However, there may be other applications where an exchange of the battery is contemplated.

In a further aspect of the present invention, a medical device for detecting at least one analyte in a body fluid is disclosed.

The term “body fluid” as used herein is a broad term and is to be given its ordinary and customary meaning to a person of ordinary skill in the art and is not to be limited to a special or customized meaning. The term specifically relates to an arbitrary fluid which typically is present in a body or body tissue of a user or a patient and/or which may be produced by the body of the user or the patient. As an example for body tissue, interstitial tissue may be named. Thus, as an example, the body fluid may be selected from the group consisting of blood and interstitial fluid. However, additionally or alternatively, one or more other types of body fluids may be used, such as saliva, tear fluid, urine or other body fluids. During detection of at least one analyte, the body fluid may be present within the body or body tissue.

The term “medical device” as used herein is a broad term and is to be given its ordinary and customary meaning to a person of ordinary skill in the art and is not to be limited to a special or customized meaning. The term specifically may refer, without limitation, to an arbitrary element or article being configured for use in the field of medical technology, specifically in the field of medical analytics or medical diagnostics. The medical device may be configured for performing at least one medical function and/or for being used in at least one medical process, such as one or more of a therapeutic process, a diagnostic process or another medical process. The medical device may be configured to be mounted on a skin site of an extremity of the user. The extremity may be selected from the group consisting of: an arm, specifically an upper arm; a stomach; a shoulder; a back; hip; a leg. Specifically, the extremity may be the upper arm. However, also other applications may be feasible. The medical device may comprise at least one component which may be configured to stay outside of the body tissue. Further, the medical device may comprise, as outlined above, the at least one invasive portion. The invasive portion may be configured for being inserted into the body tissue of the user.

The medical device comprises:
at least one analyte sensor having an insertable portion adapted for at least partially being inserted into a body tissue of a user;
at least one insertion component configured for inserting the analyte sensor into the body tissue of the user; and
at least one electronics unit, wherein the analyte sensor is operably connected to the electronics unit, wherein the electronics unit comprises the circuit carrier assembly as described above or as will further be described below in more detail.

For possible definitions and options of the circuit carrier assembly reference may be made to the disclosure of the circuit carrier assembly according to the present invention.

The analyte sensor for detecting at least one analyte in a body fluid of a user, such as in a body fluid contained in a body tissue of the user, may be configured for being used in qualitatively and/or quantitatively detecting the at least one analyte. The term “analyte” as used herein is a broad term and is to be given its ordinary and customary meaning to a person of ordinary skill in the art and is not to be limited to a special or customized meaning. The term specifically may refer, without limitation, to a chemical and/or biological substance which takes part in the metabolism of the body of the user. Specifically, the analyte may be a metabolite or a combination of two or more metabolites. As an example, the analyte may be selected from the group consisting of: glucose, lactate, triglycerides and cholesterol. Still, other analytes or combinations of two or more analytes may be detected. The body tissue specifically may be or may comprise fatty tissue and/or interstitial fluid. Other types of body tissue, however, are feasible. The term “analyte sensor” as used herein is a broad term and is to be given its ordinary and customary meaning to a person of ordinary skill in the art and is not to be limited to a special or customized meaning. The term specifically may refer, without limitation, to a sensor which is capable of qualitatively or quantitatively detecting the presence and/or the concentration of the at least one analyte. The analyte sensor may be an electrochemical analyte sensor. The analyte sensor may comprise at least two electrodes. Specifically, the analyte sensor may comprise at least one two-electrode sensor. The two-electrode sensor may comprise precisely two electrodes, such as a working electrode and at least one further electrode such as a counter electrode, in particular a working electrode and a combined counter/reference electrode. Specifically, the analyte sensor may be a needle-shaped or a strip-shaped analyte sensor having a flexible substrate and the electrodes disposed thereon. As an example, the analyte sensor may have a total length of 5 mm to 50 mm, specifically a total length of 7 mm to 30 mm.

The term “insertion component” as used herein is a broad term and is to be given its ordinary and customary meaning to a person of ordinary skill in the art and is not to be limited to a special or customized meaning. The term specifically may refer, without limitation, to an arbitrary element which may be insertable at least partially into a body tissue, particularly in order to deliver or to transfer a further element. The insertion component may specifically be configured for supporting an insertion of the analyte sensor for detecting at least one analyte in a body fluid. The analyte sensor may remain in the body tissue of the user for the predetermined period of time whereas the insertion component may be removed from the body tissue after insertion of the analyte sensor. However, alternatively, embodiments may be feasible wherein the analyte sensor as well as the insertion component may remain in the body tissue of the user for the predetermined period of time. The insertion component may comprise a tip or a sharp end for inserting the analyte sensor into the body tissue.

The insertion component for inserting the analytical sensor into the body tissue of the user may be or may comprise an insertion cannula or an insertion needle. The term “insertion cannula” as used herein is a broad term and is to be given its ordinary and customary meaning to a person of ordinary skill in the art and is not to be limited to a special or customized meaning. The term specifically may refer, without limitation, to a hollow needle which may be at least partially slotted. The analyte sensor may be received within the insertion cannula, such as within a lumen of the insertion cannula. The term “insertion needle” as used herein is a broad term and is to be given its ordinary and customary meaning to a person of ordinary skill in the art and is not to be limited to a special or customized meaning. The term specifically may refer, without limitation, to a compact needle, specifically without a slot and without any hollow parts. The analyte sensor may be received on an outer surface of the insertion needle.

The term “electronics unit” as used herein is a broad term and is to be given its ordinary and customary meaning to a person of ordinary skill in the art and is not to be limited to a special or customized meaning. The term specifically may refer, without limitation, to a unit, such as a unit which may be handled as a single piece, which is configured for performing at least one electronic function. The electronics unit may have at least one interface for being connected to the analytical sensor, wherein the electronics unit may provide at least one electronic function interacting with the analytical sensor, such as at least one measurement function.

The electronics unit may be configured for one or more of determining and/or controlling a detection of the analyte and/or transmitting measurement data to another component. Specifically, the electronics component may be configured for one or more of performing a measurement with the sensor, performing a voltage measurement, performing a current measurement, recording sensor signals, storing measurement signals and/or measurement data, transmitting sensor signals to another component. Thus, the electronics unit specifically may comprise at least one of: a voltmeter, an ammeter, a potentiostat, a voltage source, a current source, a signal receiver, a signal transmitter, an analog-digital converter, an electronic filter, a data storage device, or an energy storage device.

The analyte sensor may be partially enclosed by the electronics unit housing. The electronics unit specifically may comprise the at least one electronics unit housing, wherein the analytical sensor, e.g. with a proximal end, may protrude into the housing and may be electrically connected with at least one electronic component within the housing. As an example, the proximal end and/or at least one contact portion of the electrochemical sensor may protrude into the electronics unit housing and, therein, may be electrically connected to the at least one electronic component, such as to at least one circuit carrier and/or at least one contact portion of the electronics unit, e.g. by one or more of a soldering connection, a bonding connection, a plug, a clamping connection or the like. The electronics unit specifically may be used as a transmitter for transmitting measurement data to at least one external device, such as to at least one receiver, e.g. wirelessly.

In another aspect of each of the assembly and the method described above, the battery clip includes side walls and ramps or hooks extending downwardly from the side walls, wherein the ramps or hooks are configured to releasably secure the battery clip to the circuit carrier.

In another aspect of each of the assembly and the method described above, the circuit carrier includes cut-out openings and one or more flexible tabs extending into the cut-out openings wherein the cut-out openings are configured to receive the side walls and the ramps or hooks of the battery clip.

In another aspect of each of the assembly and the method described above, the flexible tabs of the circuit carrier are configured to flex down during an assembly step of snapping the battery clip onto the circuit carrier as the ramps or hooks of the battery clip urge the flexible tabs downward during the battery clip insertion.

In another aspect of each of the assembly and the method described above, the flexible tabs of the circuit carrier are configured to snap into a position disposed in the openings of the side walls of the battery clip wherein the flexible tabs are releasably secured in the openings of the side walls via secure engagement with the ramps or hooks and the side walls of the battery clip to thereby releasably secure the battery clip in place on the circuit carrier.

In an aspect of each of the assembly and the method described above, the fingers of the battery clip can be configured to exert a force against a coin cell battery when a coin cell battery is received in the battery clip for securing a coin cell battery in the battery clip.

In an aspect of each of the assembly and the method described above, the battery clip can include two of the flexible conductive fingers.

In an aspect of each of the assembly and the method described above, the battery clip can comprise a folded sheet metal material.

In an aspect of each of the assembly and the method described above, the assembly can further include a coin cell battery retained in the battery clip.

In another aspect of the battery clip described above, the housing of the battery clip can further comprise at least one stopper configured for stopping a sliding movement of the battery when the battery and the circuit carrier are slid into the receptacle of the housing.

In an aspect of each of the assembly and the method described above, the assembly can further include a continuous glucose monitor wherein the assembly is configured to provide electrical power to the continuous glucose monitor.

In an aspect of each of the assembly and the method described above, the assembly can further include an insulin infusion pump wherein the assembly is configured to provide electrical power to the insulin infusion pump.

In another aspect of the disclosure, a medical sensing device is provided for sensing an analyte, the medical sensing device adapted to be mounted against a skin of a patient, the medical sensing device comprising:
a case having a lower major wall adapted to be mounted against a skin of a patient, and an upper major wall opposing the lower major wall;
a sensor extending from the case and having a distal end sensitive to the analyte to produce an electrical signal, and a proximal end within the case having electrical contacts; and
the above-described circuit carrier assembly disposed within the case and supported by one of the major walls.

In an aspect, the medical sensing device can be a continuous glucose monitor.

The disclosure herein provides for a battery to be connected to a circuit carrier with no soldering needed while minimizing the thickness and footprint of the device. In other words, in medical device applications, a snap-on battery clip for a medical device connects a battery to a circuit carrier without having to solder the battery clip to the circuit carrier or the battery to the circuit carrier. Further, as the battery clip is secured to the circuit carrier, this assembly provides a self-contained connection without relying on a casing of a medical sensing device to provide a mechanical structure, thus allowing for a reduction in the thickness of the circuit carrier, the housing and the overall device. As an example, in some embodiments, the circuit carrier utilized can be a flexible printed circuit board rather than a rigid printed circuit board, which means that the board thickness as an example can typically be reduced from about 0.5 mm to about 0.12 mm. The thickness of the housing of the medical sensing device case can also be accordingly reduced to a minimum. The battery clip is configured to receive and snap onto a plurality of flexible tabs (typically two of such flexible tabs) of the circuit carrier by a simple translation movement to retain the battery clip in position on the circuit carrier. A battery (e.g., a coin cell battery) can be inserted into the battery clip. The disclosed embodiments provide a cost-effective solution and avoid a battery contact pressed between a circuit carrier and a case. In other words, known battery clips tend to place stress on the circuit carrier and the case.

The battery clips as disclosed herein are configured to press a battery and circuit carrier (e.g., a flexible circuit board) together such that the battery's first terminal contacts a first battery contact pad of the circuit carrier. The battery clip includes one or more fingers, typically two fingers, which contact the battery's second terminal and simultaneously establish an electrical connection with a second battery contact pad of the circuit carrier. The disclosed embodiments provide good contact forces that typically provide a secure and reliable connection while minimizing stresses placed on circuit carriers (circuit boards) and minimizing the footprint of the overall device. The disclosed battery clip is also scalable to adapt to future battery sizes and technologies. Further, unlike some known devices, no customized battery is needed and no customized battery is needed where the battery would have solder tabs for use in soldering the battery to the circuit carrier.

### BRIEF DESCRIPTION OF THE SEVERAL VIEWS OF THE DRAWINGS

The drawings, described below, are for illustrative purposes and are not necessarily drawn to scale. Accordingly, the drawings and descriptions are to be regarded as illustrative in nature, and not as restrictive. The drawings are not intended to limit the scope of the invention in any way. To easily identify the discussion of any particular element or act, the most significant digit or digits in a reference number refer to the figure number in which that element is first introduced.

FIG. 1 illustrates a perspective view of a battery clip according to embodiments.

FIG. 2A illustrates a perspective view of a top side of a circuit carrier according to embodiments.

FIG. 2B illustrates a perspective view of a bottom side of a circuit carrier according to embodiments.

FIG. 3 illustrates a top perspective view of a circuit carrier assembly, showing a top side of a portion of a circuit carrier and showing a battery clip according to embodiments.

FIG. 4 illustrates a bottom perspective/plan view of a circuit carrier assembly, showing a bottom side of a portion of a circuit carrier and showing a battery clip according to embodiments.

FIG. 5 illustrates a top perspective view of a circuit carrier assembly in combination with a coin cell battery, showing a top side of a portion of a circuit carrier, a battery clip, and a coin cell battery according to embodiments.

FIG. 6 illustrates the same components as shown in FIG. 5, but shows a top plan view according to embodiments.

FIG. 7 illustrates a simplified plan view block diagram of a continuous glucose monitor (CGM) wireless transmitter having a circuit carrier assembly and a coin cell battery secured to a circuit carrier with a battery clip according to embodiments.

### DETAILED DESCRIPTION

Specific embodiments of the present disclosure will now be described. The invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which embodiments of this disclosure belong. The terminology used herein is for describing particular embodiments and is not intended to be limiting of the disclosure. As used in the specification and appended claims, the singular forms “a,” “an,” and “the” are intended to include the plural forms as well, unless the context clearly indicates otherwise.

Methods and systems and parts thereof described herein can be combined to implement embodiments of the disclosure.

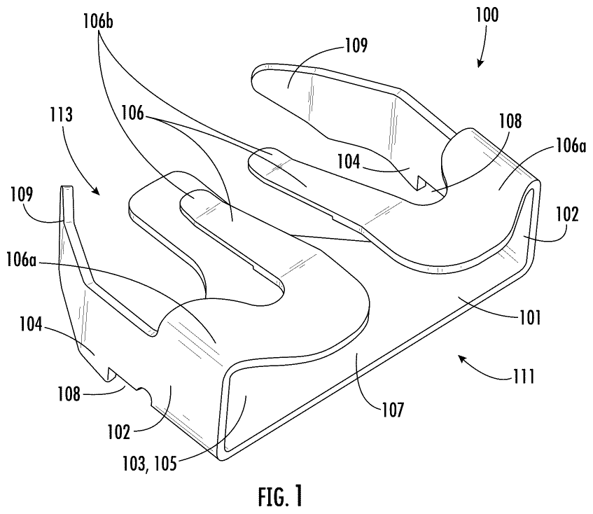

To ensure a reliable mechanical and electrical connection between a battery and a circuit carrier, circuit carrier assemblies including a circuit carrier and a battery clip are disclosed in accordance with several embodiments, as will be explained in greater detail below in connection with FIGS. 1-7.

FIG. 1 shows a battery clip 100 for retaining at least one battery 502 (not shown in FIG. 1), specifically at least one coin cell battery, to at least a first battery contact pad of a circuit carrier 200 (also not shown in FIG. 1) according to the present invention in a perspective view.

The battery clip 100 is configured to be secured to a circuit carrier 200 which can include or consist of a printed circuit board (PCB) comprising a rigid PCB, flexible PCB, or a combination of a rigid and flexible PCB. The battery clip 100 is configured to retain a battery 502 (e.g., a coin cell battery) for providing power to the circuit carrier as further described herein and below. The battery clip 100 comprises at least one housing 101. The housing 101 comprises at least one receptacle 103 for at least partially receiving the circuit carrier and the battery. The receptacle 103 comprises at least one opening 105 through which the battery and the circuit carrier are at least partially insertable. The terms “retain” and “securely retain” are used interchangeably herein to refer to the ability of the battery clip to hold a battery so that it is free from loss and provide reliable electrical connections between the battery and the circuit carrier to provide power. The battery clip is configured to be releasably attached to the circuit carrier.

A flexible printed circuit board (PCB) may include an assembly of electronic circuits and/or components surface mounted on a flexible plastic substrate, for example. The flexible plastic substrate may be, e.g., a polyimide, polyether ether ketone (PEEK), a conductive transparent polyester film or the like. Flexible PCBs are typically very thin, usually no more than a few millimeters thick. A flexible PCB can advantageously bend or flex during its use. In contrast, rigid PCBs, which are thicker than flexible PCBs, may break and/or the circuitry imprinted thereon may malfunction if they are bent or flexed during use. A small battery-operated electronic device that may benefit from having a flexible PCB is a continuous glucose monitor (CGM) wireless transmitter. A CGM wireless transmitter may be placed on a user's body to automatically take glucose measurements at regular intervals and wirelessly transmit those measurements to a receiver and/or an insulin pump. A CGM wireless transmitter with a flexible PCB for a sensor and wireless transmitter circuitry may allow the CGM wireless transmitter to conform to the surface of a user's body at the attachment site, thus improving the CGM wireless transmitter's adhesion thereto and/or the user's comfort while wearing the CGM wireless transmitter. CGMs can be powered by coin cell batteries, such as, e.g., miniature silver oxide batteries. However, coin cell batteries held in conventional coin cell battery holders, which are generally configured for mounting to rigid PCBs, would defeat the purpose of having a flexible PCB because the thickness and size of the battery holders would add rigidity to the flexible PCB. A conventional coin cell battery holder made of plastic and metal connections would also increase the volume needed to connect the battery to the board, thereby making the device bigger. Generally, when the disclosed battery clip is assembled with a flexible circuit carrier, the flexible circuit carrier will generally retain its flexibility since some of it is typically secured between two generally rigid housing bodies and cannot move but the part of the circuit carrier not physically constrained by such housing bodies is still flexible. Such assemblies generally have good performance in use because during any vibrations during use or drops by a user, any movements of the battery will not be translated as a stress in the circuit carrier because the circuit carrier in this example embodiment is flexible. However, this same concept also works well when the disclosed battery clip is used with a rigid circuit carrier as long as the rigid circuit carrier is not placed under a stress which is higher than it can withstand.

In accordance with the embodiments disclosed herein, coin cell batteries (and batteries with a similar configuration) may be securely mounted to a circuit carrier, e.g., a flexible PCB, by using the battery clip disclosed herein. Such mounting maintains the overall flexibility of a flexible PCB. For example, in one embodiment, a flexible PCB having a coin cell battery mounted thereon using the battery clip disclosed herein may provide for a CGM patch having a thickness typically about 0.5 mm greater than a similar patch having a battery soldered on the PCB, while providing a lower manufacturing cost and the ability to replace the battery with a new one. Further, the embodiments described herein provide a CGM wireless transmitter with a flexible PCB such that the CGM wireless transmitter can readily conform to the surface of a user's body at the attachment site.

The battery clip 100 comprising its housing 101 may be manufactured as one single piece. Battery clip 100 is typically made of a metallic material, preferably a folded sheet metal, e.g., a folded stainless steel sheet material, more preferably a folded, heat-treated stainless steel sheet material which is optionally plated and preferably plated with nickel to provide improved electrical connections and/or to avoid galvanic corrosion in use. Although the battery clip could be made with other conductive materials, a metal is typically stiffer than non-metallic materials, thereby making for a thinner clip when trying to provide for a clip to achieve certain desired contact forces.

In specific embodiments, the housing 101 of the battery clip 100 is configured to include two opposing side walls 102 which are typically perpendicular to one another, a transversely extending bottom wall 107 which is connected to each of the side walls and extends from one side wall to the opposing side wall, openings 108 in the side walls 102, and two ramps or hooks 104 extending downwardly from side walls 102, wherein the ramps or hooks 104 are typically positioned adjacent the openings 108 and configured to releasably secure battery clip 100 to the circuit carrier (described in further detail below). The housing 101 of the battery clip 100 further includes at least one flexible conductive finger 106, preferably two flexible conductive fingers 106, which include proximal portions 106a extending away from side walls 102 and inwardly toward one another and each terminating in a distal end portion 106b. The distal end portion 106b of the at least one flexible conductive finger 106 is configured to push down on and create electrical communication with a coin cell battery once the battery is inserted in the battery clip 100. The at least one flexible conductive finger 106 forms a top wall of the housing 101 of the battery clip. The housing 101 of the battery clip may be manufactured as one single piece.

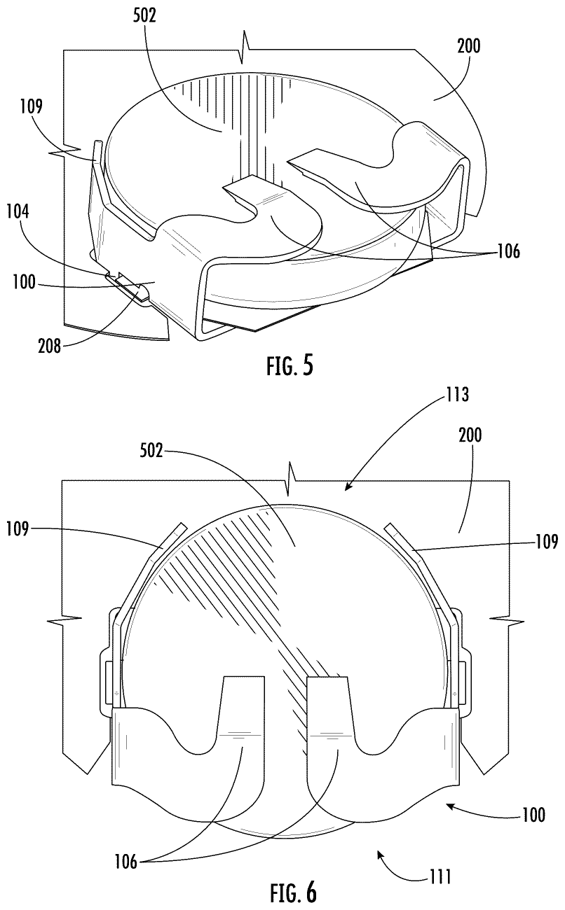

The housing 101 may further comprise at least one stopper 109. The stopper 109 may be configured for stopping a sliding movement of the battery when the battery and the circuit carrier are slid into the receptacle 103 of the housing of the battery clip. The housing 101 may have at least one first side 111 and at least one opposing second side 113. The opening 105 may be located at the first side 111 and the stopper 109 may be located at the second side 113. Specifically, the stopper 109 may extend from one or both of the sidewalls 102 and extend inwardly and be configured to provide an abutting surface for retaining the battery at the second side 113 once the battery is assembled with the battery clip (see also FIGS. 5 and 6).

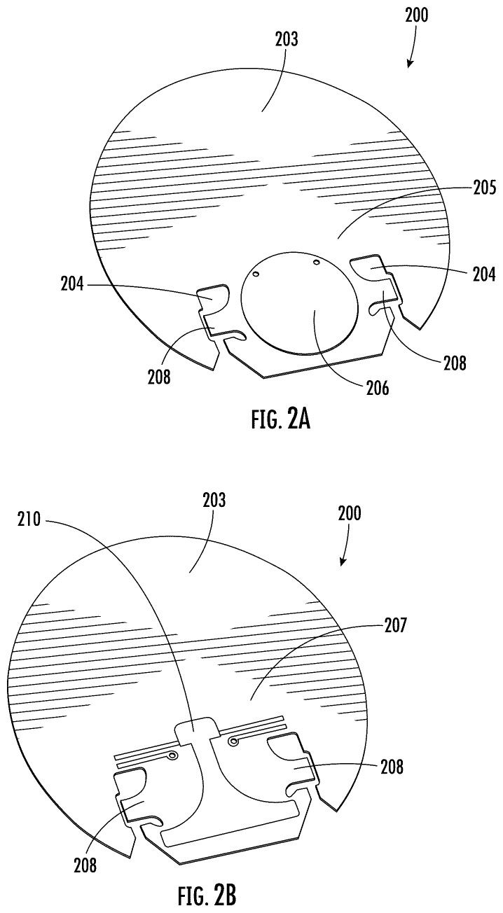

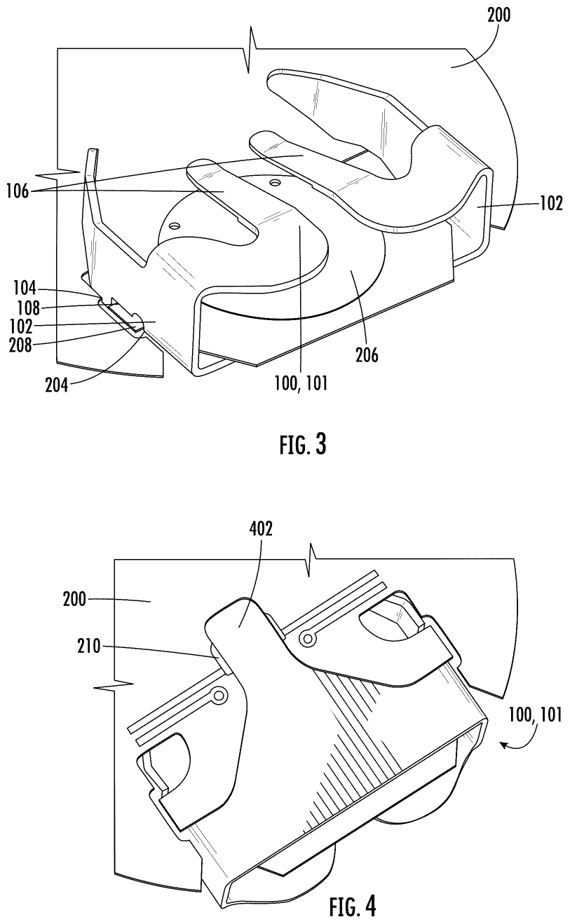

FIG. 2A illustrates a top perspective view of a circuit carrier 200. The circuit carrier 200 comprises at least a first battery contact pad 206. The circuit carrier 200 is configured to be at least partially insertable into the receptacle 103 of the housing 101 of the battery clip 100. The circuit carrier 200 may be or may comprise a printed circuit board 203. In the drawing figures, the circuit carrier 200 is illustrated schematically. Thus, details of the circuit carrier 200 are not depicted. Circuit carrier 200 includes cut-out openings 204, the first battery contact pad 206 on its top side 205, and includes one or more flexible tabs 208 extending into the cut-out openings 204. The cut-out openings 204 may respectively extend from an outer edge of the circuit carrier 200, specifically towards an interior of the circuit carrier 200, and they are sized and provided such that they are configured to receive the ramps or hooks 104 and side walls 102 of the battery clip 100 (see FIGS. 3-6). In an aspect, two of the flexible tabs 208 can be provided. The one or more flexible tabs 208 are sized and provided such that they are configured to releasably secure with and between the ramps or hooks 104 and side walls 102 of the battery clip 100. When battery clip 100 is secured to circuit carrier 200, the flexible tabs 208 of the circuit carrier 200 are adapted to flex down during insertion of the battery clip 100 and then snap back into position to thereby releasably secure battery clip 100 in place on the circuit carrier 200. The first battery contact pad 206 comprises an electrically conductive material and can comprise a gold plating material disposed on a copper trace.

FIG. 2B illustrates a bottom perspective view of circuit carrier 200 and shows its bottom side 207. Circuit carrier 200 includes a second battery contact pad 210 disposed on its bottom side. Battery contact pads 206 and 210 typically have opposite polarities with one of them being a positive terminal and the other being a negative terminal.

FIG. 3 illustrates in a top view a circuit carrier assembly comprising battery clip 100 releasably secured to circuit carrier 200. Side walls 102 of battery clip 100 are received in the cut-out openings 204 of the circuit carrier 200. The flexible tabs 208 of the circuit carrier 200 are adapted to flex down during an assembly step of snapping the battery clip 100 onto the circuit carrier 200 as the ramps or hooks 104 of the battery clip 100 urge the flexible tabs 208 downward during the battery clip insertion assembly step. Thereafter, the flexible tabs 208 of the circuit carrier snap back into a position disposed in the openings 108 of the side walls 102 wherein the flexible tabs 208 are releasably secured in the openings 108 via secure engagement with the ramps or hooks 104 and side walls 102 of the battery clip 100 to thereby releasably secure the battery clip 100 in place on the circuit carrier 200. The circuit carrier assembly is configured to clamp the battery and the circuit carrier together in a sandwich structure to securely hold the battery in the assembly and provide electrical communication between the battery and the circuit carrier.

FIG. 4 illustrates in a bottom view the circuit carrier assembly comprising battery clip 100 releasably secured to circuit carrier 200 as described above. In this view, one can see that a rearwardly extending lower wall portion 402 of the battery clip 100 is in physical contact with the second battery contact pad 210 to establish electrical communication between the battery clip 100 and the second battery contact pad 210.

FIG. 5 and FIG. 6 are similar to the view shown earlier in FIG. 3, but each shows the circuit carrier assembly with a battery 502 secured thereto. The battery 502 may specifically be a coin cell battery. It can be seen in FIGS. 5 and 6 how flexible conductive fingers 106 press down on the top of the battery 502 to establish an electrical communication between fingers 106 of the battery clip and the battery 502.

The battery clip 100 is configured for retaining the battery 502 to the first battery contact pad 206 of the circuit carrier 200. The battery clip is configured for contacting the battery 502 being arranged on the top (first) side 205 of the circuit carrier 200 as the battery is inserted into the receptacle 103 of the battery clip, and the rearwardly extending lower wall portion 402 of the housing of the battery clip is configured for contacting the bottom (second) side 207 of the circuit carrier 200 being inserted into the receptacle 103, the bottom side 207 opposing the top side 205. The top side 205 may also be referred to as a battery-facing side of the circuit carrier. The top side 205 may also be referred to as a front side of the circuit carrier and the bottom side 207 may be referred to as an opposing rear side of the circuit carrier. The top side 205 and the bottom side 207 may extend essentially parallel to each other. The battery clip is thus configured for providing force onto the battery 502, specifically from two opposite directions. Thus, the battery clip is configured to press on the battery to cause the first terminal of the battery to physically contact and be in electrical communication with the first battery contact pad of the circuit carrier.

As should be clear from the above description, the one or more flexible conductive fingers 106 of the battery clip make contact with and establish electrical communication with a terminal of the battery 502, and the housing of the battery clip is conductive and its rearwardly extending lower wall portion 402 (i.e., its bottom wall) is configured to establish electrical communication with the second battery contact pad 210 disposed on the bottom side of circuit carrier 200 to thereby establish electrical communication between the battery and the second battery contact pad 210 (see FIGS. 2B and 4). As is apparent from the above discussion and as shown in FIGS. 5 and 2A, once battery 502 is inserted, the bottom of battery 502 is thereby pressed down on the first battery contact pad 206 to establish electrical communication there between. The battery 502 may be at least partially insertable into the receptacle 103 of the housing 101 of the battery clip 100 and may specifically be a coin cell battery.

The battery clip is configured to be secured to the circuit carrier, wherein the battery clip is configured to securely retain the battery for providing power to the circuit carrier and wherein the battery clip is configured to act as a C clamp, thereby forming a sandwich with the battery and the circuit board and providing a clamping force to ensure a good electrical contact. The clamping force is provided by the at least one flexible conductive finger against the battery. A preloading of the at least one flexible conductive finger is provided as a result of the battery insertion and an interference fit between the at least one flexible conductive finger and the battery is typically established. Thus, the at least one flexible conductive finger is configured to exert a force against the battery when the battery is received in the receptacle of the battery clip for securing the battery in the battery clip.

As should also be apparent, once the first and second pads of circuit carrier 200 are in proper electrical communication with the respective terminals of the coin cell battery, the first and second pads of circuit carrier 200 can supply electrical power to the electrical circuitry of the circuit carrier 200 which in turn can provide electrical power to an associated medical device like a continuous analyte monitor (e.g., a continuous glucose monitor) and/or a medication delivery system (e.g., an insulin infusion pump).

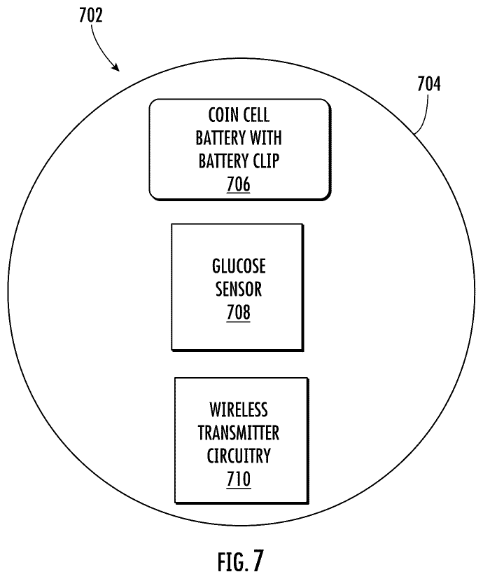

FIG. 7 illustrates a continuous glucose monitor (CGM) 702 having a circuit carrier 704 like the circuit carriers described above in accordance with one or more embodiments. The CGM 702 includes circuit carrier 704 having one or more coin cell battery with battery clips 706 mounted thereon. One or more coin cell battery with battery clips 706 may be mounted on and electrically connected to circuit carrier 704 in the manner shown in the preceding Figures and as described above. The continuous glucose monitor (CGM) 702 also includes a continuous glucose monitor sensor 708 and wireless transmitter circuitry 710 each fabricated on top surface (or alternatively on a bottom surface) of circuit carrier 704 and electrically connected to each other, to one or more coin cell battery with battery clips 706, and to possibly other circuits or components (not shown). A portion of glucose sensor 708 is inserted into the skin of a user's body and may be configured to continually measure glucose levels, and wireless transmitter circuitry 710 may be configured to wirelessly transmit those glucose measurements to a CGM receiver, an insulin pump, a diabetes manager and/or an app for controlling one or much such devices or monitoring data from such devices. The proximal end of the sensor may be affixed to the circuit carrier 704. Other circuits and circuit components (not shown) may also be fabricated on circuit carrier 704. Advantageously, the continuous glucose monitor (CGM) 702 is capable of bending and/or flexing such that it may conform to a surface of the user's body to which it is attached, improving the adherence of the continuous glucose monitor (CGM) 702 to the skin surface and/or the user's comfort while wearing the continuous glucose monitor (CGM) 702. The transmitter transmits the sensor signals to a monitoring device, such as a handheld analyte monitor, which may have a display, or a smartphone.

The CGM 702 can be housed in a single housing or case to form a CGM patch (an overall device) which can be secured to a patient's body and include the above-described sensor that extends into the patient's body. The case may comprise an upper housing including an upper major wall and a lower housing including a lower major wall wherein the upper major wall opposes the lower major wall. The upper housing is typically connected to the lower housing in a water-tight manner. The lower major wall is typically adapted to be mounted against the skin of a patient. In an embodiment, the upper housing may be ultrasonically welded to the lower housing. An analyte (e.g., CGM) sensor can extend from the case and have a distal end sensitive to the analyte to produce an electrical signal, and a proximal end within the case having electrical contacts. The above-described circuit carrier assembly can be disposed within the case and supported by one of the major walls.

In embodiments, the CGM patch is smaller than many other CGM patches. For example, the CGM patch may be from about 3.8 mm to about 4.4 mm thick and have a footprint of about 56 mm² to about 132 mm², each of which is generally lower than other known CGM patches.

With reference to FIG. 7, in another embodiment, a medication delivery system can be provided by substituting the CGM and glucose sensor with corresponding insulin delivery components. In an embodiment, the medication delivery system can be an insulin delivery system, and the medication delivery device can be an insulin delivery device, e.g., an insulin pump, typically an insulin patch pump.

In yet another embodiment, with reference to FIG. 7, a blood glucose monitoring system can be provided by substituting the CGM and glucose sensor with corresponding blood glucose monitoring components.

### LISTING OF DRAWING ELEMENTS

100 battery clip
101 housing
102 side wall
103 receptacle
104 ramp or hook
105 opening
106 flexible conductive finger
106a proximal portion
106b distal end portion
107 bottom wall
108 opening
109 stopper
111 first side
113 second side
200 circuit carrier
203 printed circuit board
204 cut-out openings
205 top side
206 first battery contact pad
207 bottom side
208 flexible tab
210 second battery contact pad
402 lower wall portion
502 battery
702 continuous glucose monitor (CGM)
704 circuit carrier
706 coin cell battery with battery clip
708 continuous glucose monitor sensor
710 wireless transmitter circuitry

It should be noted that the drawings are not drawn to scale, and particularly the cross-sectional views are not drawn to scale.

Having shown and described various versions in the present disclosure, further adaptations of the methods and systems described herein may be accomplished by appropriate modifications by one of ordinary skill in the art without departing from the scope of the present disclosure. Several of such potential modifications have been mentioned, and others will be apparent to those skilled in the art.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A battery clip for retaining at least one battery to at least a first battery contact pad of a circuit carrier, wherein the battery clip comprises at least one housing comprising an electrically conductive material, wherein the housing comprises opposing side walls connected to a bottom wall and at least one flexible conductive finger forming a top wall, the housing comprising at least one receptacle for at least partially receiving the circuit carrier and the battery, wherein the receptacle comprises at least one opening through which the battery and the circuit carrier are at least partially insertable, the at least one flexible conductive finger and the bottom wall being configured for retaining the battery to the first battery contact pad of the circuit carrier, wherein the at least one flexible conductive finger is configured for contacting the battery being arranged on a first side of the circuit carrier being inserted into the receptacle, wherein the bottom wall is configured for contacting a second side of the circuit carrier being inserted into the receptacle, the second side opposing the first side, and wherein the battery clip is configured to press on the battery to cause a first terminal of the battery to physically contact and be in electrical communication with the first battery contact pad.

2. The battery clip of claim 1 wherein the at least one flexible conductive finger is configured to contact a second terminal of the battery to establish electrical communication between the at least one flexible conductive finger and the second terminal of the battery, and wherein the bottom wall of the housing of the battery clip is configured to contact a second battery contact pad of the circuit carrier to establish electrical communication between the battery and the second battery contact pad.

3. The battery clip of claim 1 wherein the battery clip comprises two of the flexible conductive fingers.

4. The battery clip of claim 1 wherein the battery clip comprises a folded sheet metal material.

5. The battery clip of claim 1 wherein the battery clip includes ramps or hooks extending downwardly from the side walls, wherein the ramps or hooks are configured to releasably secure the battery clip to the circuit carrier.

6. The battery clip of claim 1 wherein the at least one flexible conductive finger is configured to exert a force against the battery when the battery is received in the receptacle of the battery clip for securing the battery in the battery clip.

7. The battery clip of claim 1 wherein the housing further comprises at least one stopper configured for stopping a sliding movement of the battery when the battery and the circuit carrier are slid into the receptacle of the housing.

8. A circuit carrier assembly comprising: the battery clip of claim 1 and a circuit carrier comprising a first battery contact pad, a second battery contact pad and a plurality of flexible tabs wherein the battery clip is configured to clamp the battery and the circuit carrier together in a sandwich structure to securely hold the battery in the assembly and provide electrical communication between the battery and the circuit carrier.

9. The circuit carrier assembly of claim 8 wherein the circuit carrier includes cut-out openings and one or more flexible tabs extending into the cut-out openings wherein the cut-out openings are configured to receive the side walls and the ramps or hooks of the battery clip.

10. The circuit carrier assembly of claim 9 wherein the flexible tabs of the circuit carrier are configured to flex down during an assembly step of snapping the battery clip onto the circuit carrier as the ramps or hooks of the battery clip urge the flexible tabs downward during the battery clip insertion.

11. The circuit carrier assembly of claim 10 wherein the flexible tabs of the circuit carrier are configured to snap into a position disposed in the openings of the side walls of the battery clip wherein the flexible tabs are releasably secured in the openings of the side walls via secure engagement with the ramps or hooks and the side walls of the battery clip to thereby releasably secure the battery clip in place on the circuit carrier.

12. The circuit carrier assembly of claim 8 further comprising a coin cell battery retained in the battery clip.

13. The circuit carrier assembly of claim 8 further comprising a continuous glucose monitor wherein the assembly is configured to provide electrical power to the continuous glucose monitor.

14. The circuit carrier assembly of claim 8 further comprising an insulin infusion pump wherein the assembly is configured to provide electrical power to the insulin infusion pump.

15. A medical sensing device for sensing an analyte, the medical sensing device adapted to be mounted against a skin of a patient, the medical sensing device comprising:
a case having a lower major wall adapted to be mounted against a skin of a patient, and an upper major wall opposing the lower major wall;
a sensor extending from the case and having a distal end sensitive to the analyte to produce an electrical signal, and a proximal end within the case having electrical contacts; and
the circuit carrier assembly of claim 8 disposed within the case and supported by one of said major walls.

16. The medical sensing device of claim 15 wherein the device is a continuous glucose monitor.

17. A method for retaining a battery in a medical device, comprising: providing a circuit carrier comprising a first battery contact pad, a second battery contact pad and a plurality of flexible tabs; and providing a battery clip configured to be secured to the circuit carrier, wherein the battery clip is configured to retain the battery for providing power to the circuit carrier such that the battery and the circuit carrier are pressed together such that a first terminal of the battery physically contacts and is in electrical communication with the first battery contact pad, the battery clip further comprising one or more flexible conductive fingers configured to contact a second terminal of the battery to establish an electrical communication between the one or more fingers and the second terminal of the battery, and wherein the battery clip comprises an electrically conductive material and establishes electrical communication between the one or more fingers and the second battery contact pad.

18. The method of claim 17 wherein the one or more flexible conductive fingers are configured to exert a force against the battery when the battery is received in the battery clip for securing the battery in the battery clip.

19. The method of claim 17 wherein the battery clip comprises two of the flexible conductive fingers.

20. The method of claim 17 wherein the battery clip comprises a folded sheet metal material.

21. The method of claim 17 wherein the medical device comprises a continuous analyte monitor.

22. The method of claim 17 wherein the medical device comprises a medication delivery system.

23. The method of claim 17 further comprising providing a coin cell battery.

24. The method of claim 17 wherein the battery clip includes side walls and ramps or hooks extending downwardly from the side walls, wherein the ramps or hooks are configured to releasably secure the battery clip to the circuit carrier.

25. The method of claim 24 wherein the circuit carrier includes cut-out openings and one or more flexible tabs extending into the cut-out openings wherein the cut-out openings are configured to receive the side walls and the ramps or hooks of the battery clip.

26. The method of claim 25 wherein the flexible tabs of the circuit carrier are configured to flex down during an assembly step of snapping the battery clip onto the circuit carrier as the ramps or hooks of the battery clip urge the flexible tabs downward during the battery clip insertion.

27. The method of claim 26, wherein the flexible tabs of the circuit carrier are configured to snap into a position disposed in the openings of the side walls of the battery clip wherein the flexible tabs are releasably secured in the openings of the side walls via secure engagement with the ramps or hooks and the side walls of the battery clip to thereby releasably secure the battery clip in place on the circuit carrier.
